(19) United States
(12) Patent Application Publication
(10) Pub. No.: US 2025/0214958 A1
(43) Pub. Date: Jul. 3, 2025

(54) CANNABIS OIL COMPOSITIONS AND METHODS FOR PREPARATION THEREOF

(71) Applicant: Jason Jit-Sun TAN, West Leederville (AU)

(72) Inventor: William Clay HARVEY, West Leederville (AU)

(21) Appl. No.: 18/849,540

(22) PCT Filed: Mar. 22, 2023
(86) PCT No.: PCT/AU2023/050209

§ 371 Date: Sep. 23, 2024

(30) Foreign Application Priority Data
Mar. 29, 2022 (AU) 2022900795

(51) Int. Cl. C07D 311/80; C07C 37/01; C07C 37/74

(52) U.S. Cl. CPC C07D 311/80; C07C 37/01; C07C 37/74

(57) ABSTRACT

Provided is a method for preparing a cannabis oil composition, the method comprising the steps of eluting cannabinoids from dried cannabis plant material with a solvent to produce an eluate, filtering the eluate with a series of filters to produce a filtrate, evaporating the solvent by condensing from the filtrate with a distiller to extract the distillate, and purging the distillate via fractional distillation to remove residual solvent, thereby producing a cannabis extract. The method also includes the steps of performing a redox reaction of the cannabis extract for the complete removal of the water for creating CBN (Cannabinol) of various percentages depending on the timeframe in which the redox reaction is performed; and combining the cannabis extract with an essential oil or a carrier oil to produce the cannabis oil composition.

### TECHNICAL FIELD

This invention broadly relates to cannabis oils and cannabis oil compositions, in general, and more specifically to a unique cannabis oil extraction method using preferred solvent ethanol and cannabis oil formulations including cannabis oil compositions with a carrier oil and associated methods of preparation.

### BACKGROUND ART

The following discussion of the background art is intended to facilitate an understanding of the present invention only. The discussion is not an acknowledgement or admission that any of the material referred to is or was part of the common general knowledge as at the priority date of the application.

The endocannabinoid system within the human body is a naturally forming system that regulates a wide variety of physiological processes, such as appetite, memory, sleep, mood, pain sensation, and immune response. When a deficiency or other defect occurs, it can often lead to disease. When cannabis oil compositions are ingested, the cannabinoids immediately bind to the endocannabinoid receptors, CB1 (Brain) and CB2 (immune system) and the releasing cannabinoids are continually binding to areas within the endocannabinoid system that need healing to maximize programmed cell death.

The biological processes for the reception and interaction of the compounds found within cannabis, by the endocannabinoid system, encompasses cannabinoids found naturally in our bodies, (e.g.: 2-AG and Anandamide). These natural cannabinoids released by the body are broken down quicker, having a lesser effect than those cannabinoids extracted from cannabis. Depending on the ratio/strength of cannabinoids ingested, they will have different effects on the body and for this reason, different ratios are formulated to treat different illnesses. Scientists estimate there to be over eighty unique cannabinoids within a cannabis plant which are highly beneficial for the human body.

Bioactive extracts from Cannabis plant prepared and formulated according to existing methods may contain undesired compounds in addition to or other than THC (tetrahydrocannabinol). Medicaments containing such cannabinoids are generally not stable due to unwanted side chemical reactions such as hydroquinones formation, which may lead to a medical patient not receiving an accurate dose.

The current invention was conceived with these shortcomings in mind in an attempt to propose possible solutions, at least in part, to the known shortcomings in the art of conventional cannabis oil composition preparation practices.

### SUMMARY OF THE INVENTION

According to a first aspect of the invention there is provided a method for preparing a cannabis oil composition, said method comprising the steps of:
eluting cannabinoids from dried cannabis plant material within a solvent to produce an eluate via refluxing for a predetermined amount of time;
filtering the eluate with a series of filters to produce a filtrate;
evaporating the solvent from the filtrate by condensing with a distiller to extract a distillate;
purging the distillate via fractional distillation to remove residual solvent to produce a cannabis extract;
performing a redox reaction of the cannabis extract to remove water therefrom to create CBN (Cannabinol) via degradation of (THC) Tetrahydrocannabinol at a percentage concentration dependent on said predetermined amount of time of refluxing; and
combining the cannabis extract at the percentage concentration with an essential or carrier oil to produce the cannabis oil composition.

In an embodiment, the cannabis plant material is selectable from a non-exhaustive group consisting of cannabis indica plant, ruderalis plant, sativa plant and/or a hybrid plant.

In an embodiment, the solvent is selectable from a non-exhaustive group consisting of chloroform, light ether, ethanol isopropyl alcohol, methanol, methylene chloride, benzene and denatured ethanol.

In an embodiment, the predetermined amount of time comprises a range of between 0.5 hours and 8 hours.

In an embodiment, the predetermined amount of time is determined according to a type of solvent used.

In an embodiment, the step of evaporating the solvent from the filtrate comprises a step of adding colloidal silver water to the filtrate.

In an embodiment, the step of evaporating the solvent via condensing is performed at a temperature of between 72° C. and 78° C.

In an embodiment, the step of performing the redox reaction comprises adding colloidal silver water to the cannabis extract to extend said redox reaction and/or determine said percentage concentration of the cannabis extract.

In an embodiment, the redox reaction is performed on the cannabis extract to have a pH value of between 7.2 and 8.4.

In an embodiment, the essential or carrier oil comprises hemp seed oil.

In an embodiment, the hemp seed oil is combined with the cannabis extract to be present in the cannabis oil composition in an amount between 30% (w/w) to 95% (w/w).

In an embodiment, the percentage concentration of the cannabis extract is combined with the essential or carrier oil to be present in the cannabis oil composition in an amount between 7% (w/w) to 70% (w/w).

In an embodiment, the method comprises the step of converting the CBN (Cannabinol) in the cannabis extract back to THC (Tetrahydrocannabinol) through isomerisation.

In an embodiment, the method comprises the step of removing stems and/or seeds from the dried cannabis plant material before the step of eluting within a solvent.

According to a further aspect of the invention there is provided methods for preparing a cannabis oil composition substantially as herein described and/or illustrated.

### BRIEF DESCRIPTION OF THE DRAWINGS

The description will be made with reference to the accompanying drawings in which:

FIG. 1 is a diagrammatic representation of a refluxing and condensing stage of cannabis oil composition preparation process in accordance with an aspect of the invention;

FIG. 2A-C is a diagrammatic representation of an eluate filtration stage of cannabis oil composition preparation process in accordance with an aspect of the invention;

FIG. 3 is a diagrammatic representation of a distillation stage of cannabis oil composition preparation process in accordance with an aspect of the invention;

FIG. 4 is a diagrammatic representation of a redox reaction stage of cannabis oil composition preparation process in accordance with an aspect of the invention;

FIG. 5 is a diagrammatic representation of an infusion stage of cannabis oil composition preparation process in accordance with an aspect of the invention; and

FIG. 6 is a diagrammatic representation of representative method steps of a cannabis oil composition preparation process in accordance with an aspect of the invention.

### DETAILED DESCRIPTION OF EMBODIMENTS

Further features of the present invention are more fully described in the following description of several non-limiting embodiments thereof. This description is included solely for the purposes of exemplifying the present invention to the skilled addressee. It should not be understood as a restriction on the broad summary, disclosure or description of the invention as set out above. In the figures, incorporated to illustrate features of the example embodiment or embodiments, like reference numerals are used to identify like parts throughout.

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

The term “cannabis” generally refers to plants of the genus cannabis, including Cannabis sativa, Cannabis indica, and Cannabis ruderalis. The term “species” refers to different varieties of a particular plant genus. For example, the term species can refer to different varieties of cannabis plants. Different cannabis species often exhibit distinct chemical compositions with characteristic levels of cannabinoids and terpenes, as well as other components. Differing cannabinoid and terpene profiles associated with different cannabis species can be useful for the treatment of different diseases.

The terms “treat”, “treating” and “treatment” generally refer to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom. The treatment or amelioration of symptoms can be based on any objective or subjective parameter; including, e.g., the result of a physical examination.

The term “cannabis oil” generally refers to a mixture of compounds obtained from the extraction of cannabis plants. Such compounds include, but are not limited to, cannabinoids, terpenes, terpenoids, and other compounds found in the cannabis plant. The exact composition of cannabis oil will depend on the strain of cannabis that is used for extraction, the efficiency and process of the extraction itself, and any additives that might be incorporated to alter the palatability or improve administration of the cannabis oil.

The term “cannabinoid” generally refers to a chemical compound that shows direct or indirect activity at a cannabinoid receptor. There are two main cannabinoid receptors, CNR1 (also known as CB1) and CNR2 (also known as CB2). Other receptors that research suggests have cannabinoid activity include the GPR55, GPR18, and TRPV1 receptors. The term “phytocannabinoid” refers to cannabinoids that occur in a plant species or are derived from cannabinoids occurring species. in a plant Examples of cannabinoids include, but are not limited to, tetrahydrocannabinol (THC), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), and cannabigerol monomethyl ether (CBGM).

The term “acidic cannabinoid” generally refers to a cannabinoid having one or more carboxylic acid functional groups. Examples of acidic cannabinoids include, but are not limited to, tetrahydrocannabinolic acid (THCA), cannabidiolic acid (CBDA), and cannabichromenic acid (CBCA). Acidic cannabinoids are frequently the predominant cannabinoids found in raw, fresh, greeno (i.e., unprocessed) cannabis plant material. The term “neutral cannabinoid” refers to a cannabinoid without carboxylic acid functional groups. Examples of neutral cannabinoids include, but are not limited to, THC, CBD, CBG, CBC, and CBN.

As used herein, the term “cannabinoid profile” generally refers to the number, identity, and quantity of cannabinoids in a cannabis oil preparation. Cannabis oil compositions of the invention generally contain a blend of at least two cannabis oil preparations having distinct cannabinoid profiles. In certain embodiments, cannabis oil preparations are blended to provide defined cannabinoid levels in the compositions. When a certain amount (e.g., 75%) of a particular cannabinoid (e.g., THC) is derived from a particular cannabis oil preparation (e.g., a first cannabis oil preparation), that amount of the cannabinoid is said to be “present” in the cannabinoid profile corresponding to the cannabis oil preparation. When 75% of the THC in a composition is said to be present in a first cannabinoid profile, for example, it is meant that 75% of the THC in the final composition is derived from a first cannabis oil preparation in the composition. The remaining 25% of the THC in the final composition is derived from the other cannabis oil preparations (e.g., a second or third cannabis oil preparation) in the blend. It will be appreciated that cannabinoid profiles do not remain distinct from each other, or otherwise segregated, after blending in a composition.

The term “refluxing” generally refers to boil or cause to boil, by the heating of the solutions for a specific timeframe, whilst continually cooling the vapours produced back into a liquid, such that the vapours return back to the original stock of liquid after condensing. The term “degradation” generally refers to the structural and/or chemical deterioration of a substance such as THC (Tetrahydrocannabinol) or other plant components. Degradation can include, for example, the alteration of chemical structure, oxidation state, or metal-binding properties of the substance. The term “eluate” refers to a solution that is collected after refluxing/condensing a raw and completely dried cannabis plant material, held within an extraction solvent during the process for a required timeframe. The eluate will contain dissolved cannabinoids as well as other compounds of medicinal value.

The term “filtrate” generally refers to a solution that has passed through a membrane or strainer of variable porousness or permeability to remove either particulate matter or unwanted compounds. In the methods of the invention, an eluate is passed through a series of various grades of filter to produce a filtrate. The term “distillate” refers to a solution that has been concentrated by any known means of evaporation or distillation. In the methods of the invention, the filtrate is evaporated, removing the solvent, leaving a cannabis composition or extract.

The term “fractional distillation” generally refers to a process of evaporation or otherwise removing residual solvent from the cannabis composition by the addition of a small amount of water or ice cubes. In the methods of the invention, the distillate can be condensed by the method of distillation, then removal of the distillate (solvent) from the cannabis composition. The water having a higher boiling point than the solvent, allows residual solvent to be extracted first, leaving a clean cannabis composition or extract, for further methods of cannabis oil preparation.

The term “extract” or “cannabis composition” generally refers to a solution remaining that has been dehydrated to remove residual solvent or water. In the methods of the invention, the extract is formed by evaporating the distillate by condensing and removing with any known means in the art. The term “essential oil” or “carrier oil” generally refers to a natural plant oil typically obtained through distillation via steam/water or by mechanical methods such as cold pressing and having a chemical composition and organoleptic properties (e.g., fragrance, flavour or essence) characteristic of the plant or other source from which it is extracted.

The term “supercritical extraction” generally refers to the extraction methods via a gas under pressure at ambient temperatures, e.g. CO2. CO2 extracts, for example, display some of the characteristics of both essential oils and absolutes. Like essential oils, they contain many beneficial properties, but not all of the properties which can be obtained by the use of a solvent extraction. But unlike absolutes, they are not solvent extracted and are extracts that are similar to essential oils but usually are much thicker in viscosity. They are like steam/hydro distilled essential oils in application and composition, but they contain way more volatile components than steam distilled extracts.

The term “redox reaction” generally refers to a process which contains two compounds being heated. For example, the “cannabis composition” and water. With the aid of heat, the cannabis composition oxidises (loses electrons), whilst the water (gains electrons) reducing and evaporating to atmosphere, eventually only leaving the “cannabis composition”. The term “colloidal silver water” generally refers to a water which has been produced using 99% pure silver electrodes, which have been positively and negatively attached to a battery source for a specified timeframe, to gain a certain ppm. For example, 1 litre of rainwater, with the probes within the rainwater are charged with a 12-volt battery for 1 hour to obtain 40 ppm.

Broadly, the present invention provides a unique way of extraction for cannabis oil compositions to be obtained with the preferred embodiment being a refluxing condensing method using methylated spirits or similar denatured ethanol solvent, on any cannabis species, with the preferred embodiment for the preparation of the cannabis oil compositions to be prepared with hemp seed oil for oral treatments. The dried cannabis vegetative material in the primary stage of the process is held in the solvent and by using a refluxing/condensing method and using ice to condense the vapours, not water, for a specific timeframe, enables the extraction of the cannabinoids from the cannabis vegetative material, keeping the solvent at a constant safe temperature.

The use of ice removes energy, effectively absorbing latent heat, as well as heat energy caused by the chemical reaction and lowering the surface area to near 0° C. internally, this being the area where the vapours continually condense and drop back into the solvent, keeping the solvent containing the cannabis material at a constant boiling temperature of between 72-78° C., to create an eluate. The eluate is then filtered to produce a filtrate through a series of screens, with the vegetative cannabis material now being exposed because the cannabinoids have been extracted.

Extraction of the solvent from the filtrate is then performed, by use of an additional distiller section to extract the solvent via the same refluxing/condensing method previously described with the outcome being a cannabis composition containing all of the cannabinoids, chlorophylls, waxes, sugars and a little solvent residue. Once the solvent has ceased extraction via distillation, a cannabis oil composition will remain containing all of the cannabinoids in a liquid form at 72-78° C. At this stage of the process, fractional distillation is used to remove any residue solvent by the addition of water to the cannabis oil composition and evaporation until the atmosphere has become 100% saturated within. Once at 100% saturation, the water will not evaporate and the cannabis composition/water is then put through another stage of redox reduction which is an oxidising method.

This part of the process is carried out for any specific timeframe or predetermined amount of time to create the cannabinoid (CBN-Cannabinol) of various percentages, degrading THC (Tetrahydrocannabinol). At the completion of this stage, this is then allowed to cool, forming a thick tar (cannabis composition) and in further embodiments, the method further included mixtures of the cannabis oil composition/extract with essential oils, preferably hemp seed oil, are also described with differing ratios/percentages.

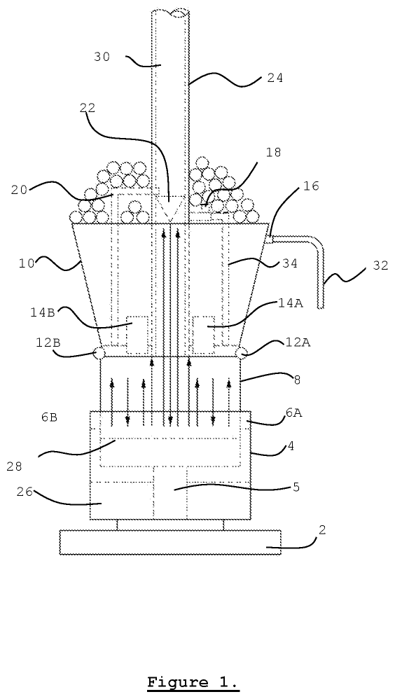

In light of this disclosure, referring now to FIG. 1 of the accompanying drawings, there is shown a refluxing and condensing stage of cannabis oil composition preparation process in accordance with an aspect of the invention. FIG. 1 shows a double boiler setup, with an ice-cooled bowl used as a lid and a copper column which houses a solid cylindrical block of ice for maximum cooling efficiency in the refluxing/condensing of the vapours created in the process, dropping back into the solvent and dried cannabis material repeatedly, e.g. over and over thousands of times, which is carried out for a specific time frame.

All components are typically made from stainless steel, copper, and brass, with everything being welded with silver solder. To enable the extraction of the cannabinoids from the cannabis material, a dual electric hot plate 2 and an extension lead are obtained. A first pot 4 is filled with water 26 to the top of the 80 mm stainless steel stand 5, which is the internal support for a second pot 8. The water is boiled continually and the continual boiled water is added to the first pot 4, maintaining a certain level. The second pot 8 holds the dry cannabis material which is totally covered with the solvent 28 to half capacity. The second pot 8 sits internally of the first pot 4, having a packing seal 6A-B around the top of the first pot 4 with a 10 mm gap for steam to vent. A PTFE garlock packing (¼′ or 6.5 mm) is used to seal between first pot 4 and the second pot 8. Any required size of PTFE Garlock packing can be used. The vents help to reduce water consumption from the continual boiling of the first pot 4 and the condensate escaping. Small amounts of water can be added through the vents needed throughout these processes. On the top of the second pot 8 containing the solvent and dried cannabis material, a tapered catchment bowl 10 with ice, which sits internally, having a ring support to stabilise and with a rubber seal 12A-B to eliminate fumes escaping is provided.

Centrally located a bored-out column support welded internally of the catchment bowl 10, with a short column 14A-B extending 100 mm above the catchment bowl's natural water level mark for vapour flow. To one side of the catchment bowl 10, a ½″ barb 16 is welded in place and a garden hose 32 is attached for excess water runoff. Ice is continually added to the catchment bowl 10 throughout the process. A ¼″ stainless hoke needle valve 18 is attached to a ¼″ tube which is welded to the short column 14A-B just above the catchment bowl's natural water level mark to relieve pressure build-up from the chemical reaction caused from the heating of the cannabis material in the solvent in the second pot 8. The ¼″ stainless steel outlet tube 34 releases into the ice-cooled water in the catchment bowl 10. Above the column support, a 100 mm copper cap 22 which is inverted is provided. The copper cap 22 is welded to a 650 mm×100 mm copper tube column 24 with a ½″ copper tube 20 welded above the inverted copper cap 22 for water drainage into the catchment bowl 10, when the ice slug melts. Internal of the inverted 100 mm copper cap 22, a condensing cone is welded. The inverted copper cap 22 fits snuggly over the column support extension which seals with the weight of the solid slug of ice 30 held in the copper tube column 24. 90 mm ice slugs are made in a 90 mm PVC stormwater pipe with a cap glued to one end, filled with water and frozen. Once frozen, they are then defrosted, removed from the casing and stored in their raw state back in the freezer until ready to use.

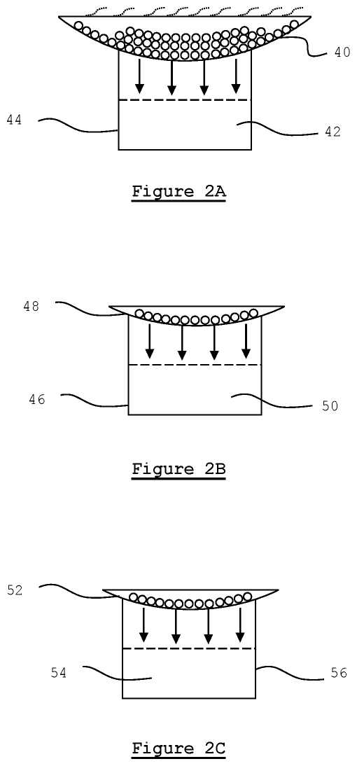

With reference now to FIG. 2A-C of the accompanying drawings, there is shown an eluate filtration stage of a cannabis oil composition preparation process in accordance with an aspect of the invention. The contents from the refluxing and condensing stage are poured into a colander 40 and an elute 42 is allowed to drip into a first catch pot 44 whilst still warm and the spent cannabis is discarded. The elute 42 is further strained into a second catch pot 46 whilst still warm through a first 35 stainless-steel mesh 48 of 500 micron or 0.5 mm, 316 grade to obtain a first filtrate 50. The first filtrate 50 is further screened through a second 400 stainless-steel mesh 52 of 37 micron or 0.037 mm to remove finer cannabis particles and obtain a second filtrate 54 in a third catch pot 56 (this step can be optional). The second filtrate 54 is poured back into the second pot 8 of refluxing and condensing stage of FIG. 1.

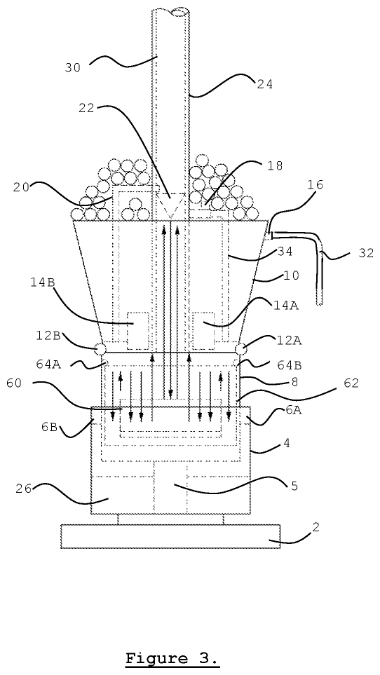

With reference now to FIG. 3 of the accompanying drawings, there is shown a distillation stage of cannabis oil composition preparation process in accordance with an aspect of the invention. FIG. 3 shows a small stainless-steel pot 60 internal of a strainer 62, which has been added to in-between the second pot 8 and the catchment bowl 10 containing ice, for evaporation of the solvent through the condensing/refluxing column, whilst still keeping the solvent at a constant temperature of 72-78° C., to eventually finish up with the distillate (solvent) being extracted and leaving behind a cannabis oil extract/water, as a filtrate. With the second filtrate now back in the second pot 8, the strainer 62 is placed with the rubber seal 64A and B already moulded to the top, internal of the second pot 8. The small stainless-steel pot 60 of approximately 1 litre is centrally located within the strainer 62, for the extraction of the solvent. The catchment bowl 10 and support ring are placed onto the top of the strainer 62. The strainer 62 has a rubber seal moulded to the top to prevent fumes from venting to the atmosphere.

The support ring is positioned under the catchment bowl 10 outer diameter, with legs extending down, hugs the outer diameter of the strainer 62 and the second pot 8 for support for the column. The 100 mm copper tube column 24 and ice slug with condensing cone is placed onto the column support extension, which is internal of the catchment bowl 10. The solvent is continually heated with the double boiler, (i.e.) boiling the water in the first pot 4 and with the valve 18 opened half a turn allowing vapour pressure from pot 8 to release into the ice water within the catchment bowl 10, with the majority of vapours condensing, and with the aid of the condensing cone, fall into the small stainless steel pot 60 centrally located within the strainer 62, which is manually removed with correct PPE, whilst some condensing vapours continue to drop back into the filtrate, continuing to reflux. The introduction of water towards the end of the solvent extraction (e.g. 100 ml), starts a process known as fractional distillation to remove any solvent residue left behind in the cannabis oil extract remaining. The fractional distillation will be continued until the atmosphere within the second pot 8 becomes fully saturated (100%) with moisture, ending this stage of the process, because the water will not evaporate.

With the preferred embodiment being with the use of ethanol, the water and the ethanol both having different boiling points, the vapour pressure of Ethanol evaporates before the water Redox reaction removing any solvent residue from the cannabis oil extract/water left behind. Once all ethanol residue is extracted, the contents from the second pot 8, being a cannabis oil extract/water combination, are added to a small stainless-steel pot of 150-250 ml, which will be placed further into a shallow mineral oil bath & heated to the required temperature to remove all traces of water from the cannabis oil extract by oxidation or the redox reaction process.

The cannabinoid oil extracts are used in methods of preparation of cannabis oil compositions for oral treatments unless CBN needs to be and (Cannabinol) formulated THC (Tetrahydrocannabinol), needs to be further reduced or completely removed. At this stage, a PH of between 7.0 And 8.3 would be achieved in the cannabis oil extract, with all chlorophylls, waxes and sugars still present when the process is completed with Ethanol only.

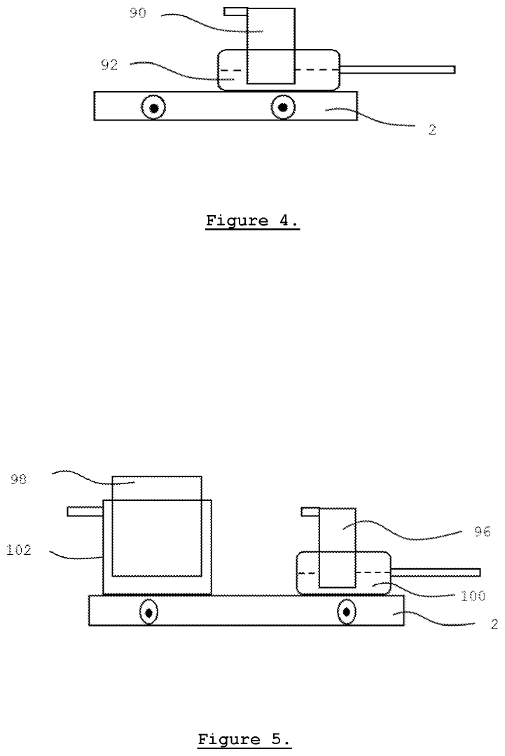

With reference now to FIG. 4 of the accompanying drawings, there is shown a redox reaction stage of cannabis oil composition preparation process in accordance with an aspect of the invention. A small stainless-steel pot 90 containing the cannabis oil extracted from the second pot 8 after the fractional distillation stage is placed in a shallow mineral oil bath 92 brought to a required temperature using the hot plate 2 to evaporate using the addition of more water or Colloidal Silver water and a longer time frame to evaporate cannabinoid i.e. (THC) Tetrahydrocannabinol until no water remains in the cannabis oil extract.

In an example embodiment, the redox reaction of cannabinoid oil extracted from cannabis indica and Cannabis sativa is performed. For the formulation of differing THC (Tetrahydrocannabinol), CBN (Cannabinol) ratios needing to be produced depends on differing timeframes of the redox reaction (i.e.) oxidation of the cannabinoid oil extract with the addition of certain amounts of water or Colloidal Silver water and by evaporation. One of the skills in the art will be able to create the ratios of the THC/CBN needed for certain medical requirements, which allow further use of these cannabinoid oil extracts of differing ratios to be use in the methods of preparation of cannabis oil compositions for oral treatment continued with the continual addition of water or Colloidal Silver water in small amounts (e.g. 10 ml) to the cannabis oil extract, and continually evaporating the water until the desired THC/CBN ratio is achieved. This can be measured by having the water in a measuring beaker for example.

The cannabis oil extract starts to smoke when the water has completely evaporated and becomes thicker to stir. Continued evaporation of the cannabis oil extract with the water addition (oxidation), degrades the THC (Tetrahydrocannabinol), until the desired THC/CBN ratio was achieved or totally CBN and other cannabinoids remain. Continued oxidation of the THC until completely exhausted, creates CBN (Cannabinol). For those skilled in the art of fluorimetry techniques, THC gives an intense greenish-yellow colour under a black light with the following reaction while CBN gives a weak reaction and CBD does not react. In an example embodiment, 0.1 g Cannabis oil extract is taken in a tube and heated to 93° C. for 5 minutes with 0.5 ml of petroleum ether or similar solvent. The solvent is evaporated on a boiling water bath to dry. 0.05 ml concentrated sulphuric acid is added after the complete evaporation of the solvent. The tube is covered and the residue/acid is mixed over a boiling water bath for 10 seconds and allowed to cool. 1 ml of 20% ammonium hydroxide is added and examined under a black light. 1 ml equates to 2 drops, when using a dropper.

One skilled in the art could use chromatography on thin layers of silica gel or alumina with a small amount of the cannabis oil extract extracted with a micropipette and placed on a plate. The plate is then placed on a small amount of solvent. The solvent is drawn to the other end via capillary action, which carries the cannabinoids present in the composition varying distances up the plate. The plate is then dried and sprayed with plant dyes which develop different colours with various cannabinoids. One skilled in the art, could use gas chromatography. In this procedure a small amount of the cannabis oil extract is injected into a long-heated coil through which gas passes and thousands of compounds leave the column and different times are detected and displayed as a series of irregular peaks shown on a gas chromatogram.

With reference now to FIG. 5 of the accompanying drawings, there is shown an infusion stage of cannabis oil composition preparation process in accordance with an aspect of the invention. Equal parts of the formulated cannabis oil extract and 95% ethanol are added together in a first beaker 96, and heated to a temperature of 78° C.-80° C. whilst stirring, the first beaker 96 is boiled in shallow water bath 100 on the hot plate 2. A second beaker 98 containing a carrier oil, for example, the hemp seed oil is also heated to 78° C.-80C° in a water bath 102. With the first beaker 96 having a dissolved solution of the desired cannabis oil extract formulated and 95% ethanol now in a liquid state and the second beaker 98 with the carrier oil at 78° C.-80° C. are blended together to form a cannabis oil composition for oral treatments.

The cannabis oil composition has a PH of between 6.5.to 7.0. One skilled in the art knows the density of the cannabis oil extract formulated for further cannabis oil compositions to be prepared, to obtain the percentages/ratios required for oral treatments. The administration of the cannabinoids a in liquid/droplet form in oral form is solely because the cannabinoids immediately bind to the receptors within the endocannabinoid system of the human system and continually bind to the areas within that system needing healing.

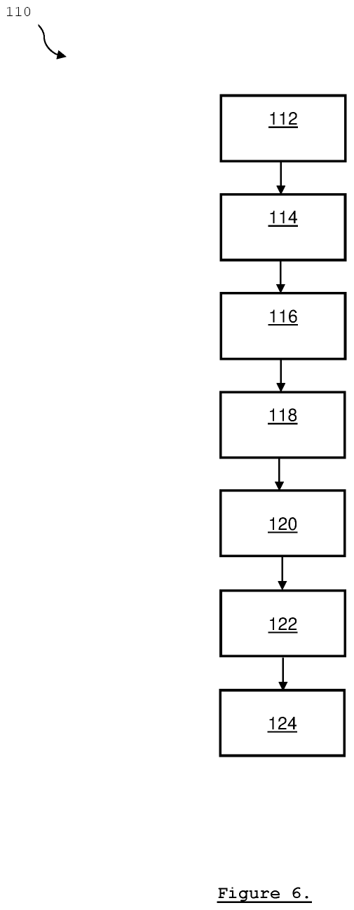

Referring now to FIG. 6 of the accompanying drawings, there is shown a flow diagram with blocks or steps representative of a method 110 for a cannabis oil composition preparation process in accordance with an aspect of the invention. The method 110 generally comprises the steps of eluting 112 cannabinoids from dried cannabis plant material held with a solvent by continually refluxing for a predetermined about of time to produce an eluate, filtering 114 the eluate with a series of filters to produce a filtrate, evaporating 116 the solvent by condensing from the filtrate with a distiller to extract the distillate, purging 118 the distillate via a fractional distillation process to remove residual solvent, thereby producing a clean, non-toxic cannabis extract, performing 120 a redox reaction of the cannabis extract for the complete removal of the water and for creating CBN (Cannabinol) of various percentages depending on the timeframe in which the redox reaction is performed by degrading the (THC) Tetrahydrocannabinol, combining 122 the cannabis extract with essential oil or carrier oil to produce the cannabis oil composition, typically in droplet form 124.

The process of preparation of the cannabis oil composition may further include an isomerisation stage. The process of converting CBD (Cannabidiol) back to THC (tetrahydrocannabinol) through the use of a dilute acid. This can then be oxidised further by the using the Redox Reaction method if needed, to create a THC (tetrahydrocannabinol)/CBN (Cannabinol) of differing ratios.

The isomerisation stage is only to be used if cannabis plants high in CBD & extremely low in THC are used. This is a costly & very time intense process which can be avoided by choosing cannabis species containing higher amounts of THC to start with, for example either the species indica or sativa or a hybrid of these two species. For the cannabis plants with a high CBD (Cannabidiol) and the composition or extract that is extracted, there's a need for isomerisation to be carried out, to convert the CBD composite back to a composite containing more TCH. Once converted to THC, this could then be oxidized through a process called the Redox Reaction as previously explained, degrading the THC by varying timeframes, until the THC is completely exhausted, creating CBN.

The isomerisation of CBD to THC could be accomplished by adding a small quantity of acid, to the oil, (cannabis composite already extracted), e.g. 10 g (cannabis composite) added to 200 ml solvent to dissolve, then simply heat with 2 ml acid for one hour. When an alcohol solvent is being used, only 2 ml of one of the following solutions could be added to every 200 ml of solvent being used: i. 120 grams of concentrated HCL added slowly to 900 ml of water, or ii. 30 ml of sulphuric acid was added slowly to 1000 ml of water. Once isomerisation is completed, the solvent is extracted using the refluxing/condensing method. Although only a small amount of diluted acid is used, neutralization with a small amount of baking soda prior to the extraction of the solvent is performed. Ice blocks are added and evaporated until the water has gone, as the solvent will disappear first.

The extraction of the Cannabinoids is to be carried out by itself or combined with isomerisation and the preparation of cannabis oil extract (thick tar), depending on the THC & CBD content and the species of cannabis being used. Large stems and seeds are removed from the completely dried cannabis plants for best results of the cannabinoids removal, with (90%-98% extractions possible), depending on the solvents being used. With fresh-picked (green) cannabis plants, the THC and CBD would be believed to be present in an acidic form, which is insoluble in some solvents and only 50% of the cannabinoids would be able to be extracted. It would be believed that a wide variety of organic and inorganic solvents could work, but toxicity, flammability, availability, and costs would need to be considered, as will be appreciated by those skilled in the art. The following are examples of typical solvents.

Chloroform—it is believed that this would be the best candidate solvent to extract close to 98% of the entire cannabinoids present in the dried cannabis material, via the entire cannabis plant extraction method, but with high toxicity and flammability being a problem.

Light Ether—it is believed that this would extract possibly 90% of the entire cannabinoids present in the dried cannabis material, via the entire cannabis plant extraction method, but also high toxicity and flammability could result.

Ethanol (grain alcohol) 95%—it is believed that this would extract possibly 90% of the entire cannabinoids present in the dried cannabis material, via the entire cannabis plant extraction method, and being the safest with low toxicity and medium flammability.

Denatured 95% ethanol—it is believed that this would extract possibly 90% of the entire cannabinoids present in the dried cannabis material, via the entire cannabis plant extraction method, and being of high toxicity/medium flammability.

Isopropyl alcohol—(rubbing alcohol is 70% isopropanol), would extract possibly 90% of the entire cannabinoids present in the dried cannabis material, via the entire cannabis plant extraction method, with medium toxicity and flammability.

Methanol—(especially industrial methanol has explosive fumes and is highly toxic), it is believed that this would extract also 90% of the entire cannabinoids present in the dried cannabis material, via the entire cannabis plant extraction method, but with high toxicity and flammability being a problem.

Methylene chloride {Dichloromethane)—it is believed that this would extract possibly 90% of the entire cannabinoids present in the dried cannabis material, via the entire cannabis plant extraction method, with no flammability and with medium toxicity

Benzene—it is believed that this would extract possibly 90% of the entire cannabinoids present in the dried cannabis material, via the entire cannabis plant extraction method, with high toxicity and highly flammable.

Common organic solvents such as benzene, chloroform, petroleum ethers etc. will not extract the water-soluble constituents of the cannabis (chlorophylls, waxes and sugars], whereas the alcohol solvents will extract these chlorophylls, waxes and sugars, as it is believed that this would be a critical part of the process to collect when extracting the cannabinoids.

The continual refluxing time, i.e. predetermined amount of time, would vary on the efficiencies of the solvents used with Chloroform and Methylene Chloride, only requiring about half an hour, whilst Ethanol, denatured ethanol or Isopropanol etcetera would need to be refluxed for a minimum of 2 hours, but for best extraction of all cannabinoids, 3-6 hours would be more beneficial, with 6 hours preferred.

Continual oxidation of the THC (Tetrahydrocannabinol) until completely exhausted, creates CBN (Cannabinol). Oxidation of cannabis oil extract through a process called the Redox Reaction to create CBN (cannabinol) can only be carried out when using the species of Cannabis Indica and Cannabis Sativa or a hybrid of these two species. The cannabis species Ruderalis is high in CBD (Cannabidiol) unless isomerisation is carried out primarily to convert CBD (Cannabidiol) back to THC (Tetrahydrocannabinol).

A PH of between 7.0-8.6 would need to be achieved for the cannabis oil extract. This would be in the form of a thick extract (thick tar once cooled), with all the chlorophylls, waxes and sugars still present. The chlorophylls, waxes and sugars still present within the cannabis oil extract, when the process is completed with an Ethanol (alcohol) are a very important part of this invention.

Using the following example, which would be used for an immune therapy working on the CB2 receptors, within the endocannabinoid system of the human body.

Residue for e.g. using a density 1.4
−1 gram divided by 1.4 (density)=0.7142857%
0.7142857%×7 grams (cannabis oil extract or tar)=4.99 ml (cannabinoid oil)
4.99 ml divided by 100 ml (hemp seed oil)=0.0499%
7 g (cannabis oil extract)+7 ml of (95% ethanol) mixed together and heated to 78-80 degrees Celsius, plus 100 ml of hemp seed oil (not the lite oil) also heated to 78-80 degrees Celsius. Once both of these ingredients are at the required temperature they would need to be infused together.

The infused ingredients provide an easy droplet form of cannabis oil composition with a PH of between 6.5 and 7.0 for oral treatments.

Cannabis oil compositions can be obtained from the entire cannabis species including hybrid species, by using only dry cannabis material, if close to all of the cannabinoids are wanting to be extracted. For the cannabis plants with a high CBD (Cannabidiol) oil extract, there's a need for isomerisation to be carried out, to convert the CBD (oil) to TCH (oil). Once converted to THC, this could then be oxidized as previously explained, degrading the THC to varying timeframes until the THC is completely exhausted, creating CBN.

In an example embodiment, 3-4 types of completely dried cannabis species of Indica/strains unknown with 140 g being blended into the second pot 8 with a total weight of (240 g or ½ lb of dried cannabis material). 3 litres of methylated spirits (denatured ethanol which is 95% ethanol and 5% methanol) are added to the second pot 8, with the cannabis material and this will just cover the cannabis with some still extruding. Both ingredients are at room temperature of 25 degree Celsius. The catchment bowl 10 and support ring onto the top of the second pot 8, making sure the taper seals perfectly within the rubber seal moulded to the top of the second pot 8, to prevent methylated spirit fumes from venting to atmosphere. The support ring sitting under the catchment bowl's outer diameter, with legs extending down, resting on the handles of the second pot 8, solely to eliminate the column from tilting and losing the sealing potential. The 100 mm copper tube column 24 is placed onto the column support extension, which is internal of the catchment bowl 10.

The ice cubes are placed into the catchment bowl 10 and an ice slug (previously made) is placed into the 100 mm copper tube column 24. The hot plate 2 is turned onto high to start the process of refluxing and condensing, allowing 6½ hours. The ¼′ Stainless Steel Hoke needle valve 18 is opened half a turn to allow pressure build-up in the second pot 8 containing (solvent/cannabis material) to vent into the bottom of the catchment bowl 10 through the ¼′ tube 34. By the time, this starts to vent, the ice would have melted and the methylated spirit fumes will vent into the water formed, dampening the fumes. It may take ½ an hour to get the whole process up and running efficiently, to maintain consistent temperatures, with ice being continually added to the catchment bowl 10 and occasionally to the copper tube column 24. A length of ½′ garden hose 32 is attached to the tapered catchment bowl's brass barb 16 to allow excess water to drain to grade or refill the now empty 90 mm PVC ice slug makers with the chilled water and freeze.

The water in the first pot 4 is boiled between 100° C. and 130° C. and with continual additions of water, the methylated spirits/cannabis is also boiled internally in the second pot 8 at between 73° C. and 78° C. The vapours caused from the chemical reaction within the second pot 8, continually condense and reflux repeatedly, i.e. over and over thousands of times for the next 6 hours to extract all of the cannabinoids, chlorophylls, sugars and waxes from the dried cannabis vegetive material. The catchment bowl 10 is continuously kept full of ice at all times throughout the process. This is another critical step, which has to be adhered to, otherwise the solvent temperature will elevate and ignite. Additional water is continuously added to the first pot throughout the process if needed.

Throughout the next 6 hours, whilst topping up the ice in the column/tapered catchment bowl 10 and water in the first pot 4, temperature readings may be externally taken with an infra-red laser thermometer gun. The temperature may be monitored and recorded. The temperature may be elevated due to the lack of ice. Good ventilation is mandatory and intrinsically safe motors are obtained if they are to be used within the vicinity. Below table 1 shows the observations to adhere to from the outside.

| Ambient temperature | 25° C. | |
|---|---|---|
| First pot | bottom | 108° C.-148° C. (Av. |
| | | 118° C.) |
| | middle | 50° C.-62° C. |
| Second pot | bottom | 42° C.-57° C. (Av. |
| | | 44° C.) |
| | top | 38° C.-50° C. (Av. |
| | | 44° C.) |
| | Internal | 70° C.-73° C. |
| | solvent | (Av.72° C.) |
| Catchment bowl | external | 10° C.-25° C. (Av. |
| | | 24° C.) |
| | internal | 4.3° C.-16° C. |
| | | (Av.14.8° C.) |
| Short column | bottom | 12° C.-19° C. (Av. |
| | | 15° C.) |
| | top | 8° C.-12° C. (Av. |
| | | 10° C.) |
| Condensing cone | | 3.6° C.-8° C. |
| | | (Av.5.6° C.) |

After 6½ hours of refluxing and condensing the hot plate 2 is turned off. The solvent/cannabis material still boils because the first pot 4 remains underneath. The cannabis material is removed from the second pot 8 and placed in the colander 40 on top of the additional Stainless Steel catch pot 44 previously set aside. The ¼″ PTFE Garlock packing in-between the first pot 4 and the second pot 8 is removed to separate the two pots. The remaining solvent/cannabis material from the second pot 8 is poured into the additional Stainless-Steel pot 44 with the colander 40 on top. The solvent is allowed to drip into the additional Stainless-Steel catch pot 44 beneath the colander 40. The cannabis materials solvent (eluate) from the Stainless-Steel catch pot 44 is poured back into the second pot 8 generally only after being through at least one of the two further filtration stages that follow.

An additional 1 litre of clean methylated spirits is poured over the cannabis material in colander 40 above stainless-steel pot 44 and then again all the cannabis materials/solvent is returned to the second pot 8 whilst still warm and but only after being further filtered through at least one of the two further filtration stages that follow. The cannabis material may be squeezed to remove the excess solvent, the spent cannabis material is discarded as most of the cannabinoids have been extracted and are in an eluate. The colander 40 is removed and the remaining contents (eluate) are strained through a 35 mesh 48, which is 316 Grade (500 micron or 0.5 mm) into the Stainless-Steel pot 46 to obtain a filtrate. The filtrate from the Stainless-Steel pot 46 is poured back into the additional Stainless Steel catch pot 56 very carefully/slowly through a 400 mesh 316 grade (27 micron or 0.037 mm). The second pot 8 is washed with water and dried. The second pot 8 is placed internally of the first pot 4 sitting on the 80 mm Stainless Steel stand 5. The ¼″ Garlock PTFE packing is installed again. The filtrate from Stainless Steel catch pot 46 or 56 is added back into the second pot 8.

A strainer 62 is placed internal of the second pot 8. A Stainless-Steel catch pot 60 of 1 litre capacity is placed central within the strainer to catch the solvent being extracted. The catchment bowl 10 and support ring are placed onto the top of the strainer. The strainer has a rubber seal 64A and B moulded to the top of it, similar to the second pot 8. The taper is sealed perfectly within the strainer to prevent methylated spirit fumes from venting to the atmosphere. The support ring sitting under the catchment bowl's outer diameter, with legs extending down, now neatly hugs the outer diameter of the strainer 62 and the second pot 8 for support for the column above. The 100 mm copper tube column 24 is placed onto the column support extension above 14A& B, which is Internal of the catchment bowl 10. Ice cubes are placed into the catchment bowl 10 and the ice slug (previously made) into the 100 mm copper tube column 24.

The hot plate 2 is turned on to start the process of distillation. The external temperatures are monitored for the next 2 hours whilst continually topping up the ice slug column 24 and the catchment bowl 10 with ice. The temperature may be monitored and recorded. The temperature may be elevated due to the lack of ice. Below table 2 shows the observations to adhere to externally and internally.

| Ambient temperature | 25° C. | |
|---|---|---|
| First pot | bottom | 122° C.-48° C. (Av. |
| | | 133° C.) |
| | middle | 50° C.-62° C. (Av. |
| | | 58° C.) |
| Second pot | bottom | 42° C. - 50° C. (Av. |
| | | 44° C.) |
| | top | 44° C.-46° C. (Av. |
| | | 44° C.) |
| | Internally | 70° C.-73° C. (Av. |
| | | 72° C.) |
| Strainer external | bottom | 58° C.-65° C. (Av. |
| | | 62° C.) |
| Catchment bowl | External | 14° C.-32° C. (Av. |
| | | 24° C.) |
| Short column | bottom | 4° C.-19° C. (Av. |
| | | 15° C.) |
| | top | 8° C.-12° C. (Av. |
| | | 10° C.) |
| Long Column | | 5° C.-8° C. (Av. |
| | | 5.6° C.) |
| Condensing cone | | 3.6° C.-8° C. (Av. |
| | | 5.6° C.) |

The methylated spirits when extracted were always at 62° C. A saucepan lid is obtained. An infra-red laser thermometer gun is used to take the internal temperatures of the solvent being extracted and the filtrate below the strainer. The long copper tube column 24 is removed with the ice slug and the saucepan lid is placed onto the 100 mm column support extension above 14A and B to suppress the solvent fumes. The catchment bowl 10 and the support ring are removed. The 1 litre Stainless Steel catch pot 60 internal of the strainer 62 containing the methylated spirits is removed and poured straight into a flask using a funnel. A temperature reading can be taken of the solvent. The strainer is quickly removed to take a temperature reading of the filtrate (optional). The temperature is around 70° C.-75° C.

The strainer with Stainless Steel pot (1 litre capacity), catchment bowl 10, and support ring are replaced. The saucepan lid from 100 mm support extension above 14A&B and internal of catchment bowl 10 is removed. Now replace the long tube column 24 with the ice slug 30 still within. Throughout the next 45 minutes, the external temperatures are monitored and ice is added to the catchment bowl 10 and ice slug column as needed. The first pot 4 is also topped up as needed. When another 45 minutes have elapsed, now a total of 90 minutes, the long copper tube column 24 is removed again with the ice slug and the saucepan lid is placed onto the 100 mm column support extension 14A&B to suppress the solvent fumes. The catchment bowl 10 and the support ring are removed. The 1 litre Stainless Steel catch pot 60 internal of the strainer containing the methylated spirits is removed and poured straight into a flask using a funnel. Approximately 112 litres of methylated spirits will have been extracted. The Stainless-Steel pot 60 (1 litre capacity), catchment bowl 10, support ring are again replaced. The saucepan lid from 100 mm support extension internal above 14A and B of catchment bowl is removed. The long tube column 24 is replaced with the ice slug 30 still within. Throughout the next 30 minutes, monitor the external temperatures and continually add ice to the catchment bowl and ice slug column as needed.

A beaker with 100 ml of distilled water is added to the second pot 8 with the filtrate to start t the fractional distillation. This is simply done by removing the long tube column 24 with the ice slug 30 still within and by pouring the 100 ml of distilled water through the 100 mm support extension 14A&B within the catch pot 10 which goes directly into the 2nd pot 8. Then replace the long tube column with the ice slug 30 still within again. Approximately 2.1 Litres of methylated spirits should have now been extracted by the end of this 30-minute timeframe. Now, with the addition of water to the 2nd pot, you will have an elevated temperature in the 1st pot 4 to sometimes 180° C. externally and the second pot 8 to 60° C. initially with boiling water venting from the 1st pot. This will eventually settle down. It is caused because both the 1st pot 4 and the 2nd pot 8now have the same boiling temperature internally with water. This will remove any solvent residue in the filtrate within 2nd pot 8, leaving only water and the cannabis oil extract. With 45 minutes of fractional distillation, the continual monitoring of the external temperatures is performed. The temperature may be monitored and recorded. The temperature may be elevated due to the lack of ice.

Below table 3 shows the observations to adhere to externally and internally.

| Ambient temperature | 25° C. | |
|---|---|---|
| First pot | bottom | 120° C.-180° C. (Av. |
| | | 150° C.) |
| | middle | 120° C.-150° C. (Av. |
| | | 150° C.) |
| Second pot | bottom | 42° C.-60° C. (Av. |
| | | 44° C.) |
| | top | 42° C.-46° C. (Av. |
| | | 44° C.) |
| | Internally | 72° C. |
| Strainer external | bottom | 47° C.-57° C. (Av. |
| | | 47° C.) |
| Catchment bowl | External | 15° C.-30° C. (Av. |
| | | 22° C.) |
| | Internal | 13° C.-13.4° C. (Av. |
| | | 13.4° C.) |
| Short column | bottom | 3°° C.-23° C. (Av. |
| | | 15° C.) |
| | top | 8° C.-19° C. (Av. |
| | | 10° C.) |
| Long Column | | 5° C.-8° C. (Av. |
| | | 6° C.) |
| Condensing cone | | 9.6° C.-10° C. (Av. |
| | | 9° C.) |

After 45 minutes of the process of fractional distillation, the vapour pressure of the methylated spirits is higher than the water, exhausting all methylated spirits residue from the filtrate and being caught in the 1 litre Stainless Steel catch pot within the strainer. The water within the second pot 8 will not evaporate any further because of its enclosure and then create an equilibrium, because the vapour space within, will become 100% saturated with moisture. The cannabis oil extract still contains water, which needs to go through a further process called the redox reaction (oxidation). A shallow frypan is filled to 80% capacity with mineral oil 92, for example engine oil.

A small Stainless Steel (150 ml 250 ml) saucepan containing the cannabis oil extract which still contains 100 ml of water carefully into a shallow frypan containing mineral oil. A redox reaction is started by reducing the water, which in turn oxidises the cannabis oil extract. The shallow frypan (mineral oil bath), with small Stainless Steel (150 ml-250 ml) saucepan 90 containing the cannabis oil extract is placed onto the hot plate 2. The hot plate 2 is turned on to a setting to achieve a temperature of 110° C.-125° C. within the mineral oil bath. The temperature can be monitored with an infra-red laser thermometer gun. Once the mineral oil bath is at the required temperature of between 110° C.-125° C., the cannabis oil extract/water will be simmered between 95° C.-106° C. The temperature is maintained for 75 minutes-90 minutes, stirring from time to time.

The cannabis oil extract will gradually become thicker to stir. The small Stainless Steel (150 ml-250 ml) saucepan 90 containing the cannabis oil extract is removed from the mineral oil bath and the hot plate 2 is turned off. A shallow water ice bath is created, to place the cannabis oil extract to cool quicker. Once the cannabis oil extract has set like tar, a teaspoon is used to dig to the bottom to make sure there is no water still present in the extract. If there is still a small amount of water in the cannabis oil extract, the small Stainless Steel (150 ml-250 ml) saucepan 90 containing the cannabis oil extract is placed back in the mineral oil bath and reheated for another 30 minutes. The redox reaction is continued until all water is exhausted. The saucepan containing the cannabis oil extract is placed in the oil bath which is still warm enough to convert the cannabis oil extract back to a liquid. Once the cannabis oil extract is back to its liquid state, it can then be placed into a glass Vial/beaker/bottle for storage or further prepared to a liquid droplet form for oral consumption.

The cannabis oil extract will contain high cannabinoid concentrations including: THC (Tetrahydrocannabinol) and CBD (Cannabidiol) and a smaller amount of CBN (Cannabinol) and CBG (Cannabigerol). Many other cannabinoids, together with chlorophylls, sugars, and waxes will be present. Approximately 50 grams or more of extract will be achieved. CBN (Cannabinol) can now be achieved from this point on in larger quantities, by continually heating the cannabis oil extract with the addition of measured amounts of colloidal silver water and/or water within the small Stainless Steel (150 ml-250 ml) saucepan 90 and placed back into the shallow fry pan (mineral oil bath).

This process can be done for another 12 hours, with careful monitoring of temperatures and constant additions of colloidal silver water and/or water being infused. At the end of this redox reaction stage the THC (Tetrahydrocannabinol) will have been converted to CBN (Cannabinol) in large quantities with very little THC (Tetrahydrocannabinol) remaining. The cannabis oil extract still being in its liquid state is weighed, then mixed with a carrier oil to obtain a cannabis oil composition used for oral consumption to the required ratio needed, with the following example set for a 5% ratio. 49 grams of the cannabis oil extract is poured into a first beaker 96. 49 grams of 95% pure Ethanol is added to the first beaker 96 and gently stirred. 700 grams of hemp seed oil is taken in a second beaker 98. The second beaker containing the hemp seed oil is placed into a deeper saucepan 102 which has been filled with water to 50% capacity and then placed on the hotplate 2 and heated to between 78° C. to 80° C. This will require a little longer to heat.

The shallow frypan 100 filled to 80% with water is placed on the hotplate 2 heated to 80° C. placing the first beaker 96 within the water. The temperatures are monitored with the use of an infra-red laser thermometer gun. Once the first beaker 96 and the second beaker 98 are at the required temperature of between 78° C. to 80° C., the contents of beaker 96 are added to the second beaker 98. This will now require stirring thoroughly, which now infuses the two liquids together. The second beaker can now be poured into a 1 litre Pyrex flask and gently shaken with a lid on. Because of the heat and the gentle shaking motion, the lid on the Pyrex flask will need to be released every 3-4 gentle shakes. The pressure is relieved appropriately. Now, the cannabis oil extract is infused with the carrier oil and is allowed to cool. This infused cannabis oil composition will have a 5% ratio of cannabinoids with a PH of 6.5 to 7.0. The cannabis oil composition is stored at room temperature and in a dark place.

Alternatively, with the use of a funnel, the infused cannabis oil composition can be placed within small pharmaceutical bottles with droppers. This makes it easy for oral treatments. With the infused cannabis oil now at approximately 5%, one drop equals to 0.05 ml or 50 mg. The dosage for the 5% cannabis oil ratio includes Days 1-3=5 drops per day (0.25 ml=250 mg), Days 4-7=8 drops per day (0.4 ml=400 mg), Days 8-11=12 drops per day (0.6 ml=600 mg), Days 12-15=15 drops per day (0.75 ml-750 mg).

The purpose of taking a large volume of low percentage cannabis oil via droplets is so the cannabinoids can attach to all of the receptors within the endocannabinoid system and also smother foreign cells within the digestive system with a high oil concentration. Examples of stronger preparations of cannabis oils can be prepared also, with smaller amounts of the carrier oil being infused, but still using the exact ratio of the proportions of cannabis extract: 95% Ethanol set out previously described with examples listed below. Weaker preparations, less than the example set out previously described are not recommended, as they are deemed useless, but by all means, can be prepared, but will have little benefits.

### EXAMPLE A (IMMUNE SYSTEM)

−1 gram divided by 1.4 (Density)=0.7142857%
0.7142857%×7 gram of extract=4.99
4.99 divided by 100 ml of (Hempseed oil)=0.0499%
4,99%

This example set above is believed to work for ovarian cancer & leukaemia severe osteoporosis, depression, spinal cancer, insomnia, Parkinson's, restless leg syndrome just to name a few examples.

### EXAMPLE B-1 (COULD BE USED FOR MULTIPLE SCLEROSIS)

1 gram divided by 1.4 (Density)=0.7142857%
0.7142857%×7 gram of extract=4.99
4.99 divided by 10 ml of (Hempseed oil)=0.499%
49.9%

### EXAMPLE B-2 (EFFECTIVE FOR MULTIPLE SCLEROSIS)

1 gram divided by 1.4 (Density)=0.7142857%
0.7142857%×7 gram of extract=4.99
4.99 divided by 7 ml of (Hempseed oil)=0.714%
71.4% (ONE DROP PER DAY OR AS REQUIRED)

### EXAMPLE C

1 gram divided by 1.4 (Density)=0.7142857%
0.7142857%×7 gram of extract=4.99
4.99 divided by 50 ml of (Hempseed oil)=0.998%
9.98%

### Example D

1 gram divided by 1.4 (Density)=0.7142857%
0.7142857%×7 gram of extract=4.99
4.99 divided by 20 ml of (Hempseed oil)=0.2495%
24.95%

Optional embodiments of the present invention may also be said to broadly consist in the parts, elements, and features referred to or indicated herein, individually or collectively, in any or all combinations of two or more of the parts, elements or features, and wherein specific integers are mentioned herein which have known equivalents in the art to which the invention relates, such known equivalents are deemed to be incorporated herein as if individually set forth.

The use of the terms “a”, “an”, “said”, “the”, and/or similar referents in the context of describing various embodiments (especially in the context of the claimed subject matter) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. As used herein, the term “and/or” includes any and all combinations of one or more of the associated listed items. No language in the specification should be construed as indicating any non-claimed subject matter as essential to the practice of the claimed subject matter.

The terms “about” and “around,” as used herein to modify a numerical value, indicate a close range surrounding that explicit value. If “X” were the value, “about X” or “around X” would indicate a value from 0.8 times. to 1.2 times, preferably a value from 0.9 times. to 1.1 times, and, more preferably, a value from 0.95 times. to 1.05 times. Any reference to “about X” or “around X” specifically indicates at least the values X, 0.95 times, 0.96 times, 0.97 times, 0.98 times, 0.99 times, 1.01 times, 1.02 times, 1.0 times, 1.04 times, and 1.05 times. Thus, “about X” and “around X” are intended to teach and provide written description support for a claim limitation of, e.g., “0.98 times”.

Any method steps, processes, and operations described herein are not to be construed as necessarily requiring their performance in the particular order discussed or illustrated, unless specifically identified as an order of performance. It is also to be understood that additional or alternative steps may be employed.

1. A method for preparing a cannabis oil composition, the method comprising the steps of:
by means of double-boiler arrangement comprising an inner pot containing dried cannabis plant material and a solvent, and an outer pot containing heated water and said inner pot, eluting cannabinoids from the dried cannabis plant material within the solvent to produce an eluate via refluxing for a predetermined amount of time by maintaining the solvent at a temperature of between 72° C. and 78° C.;
filtering the eluate with a series of filters to produce a filtrate;
evaporating the solvent from the filtrate by adding water and/or colloidal silver water to the filtrate and condensing with a distiller to extract a distillate;
purging the distillate via fractional distillation to remove residual solvent to produce a cannabis extract;
performing a redox reaction of the cannabis extract to remove water therefrom to create CBN (Cannabinol) via degradation of (THC) Tetrahydrocannabinol at a percentage concentration dependent on said predetermined amount of time of refluxing; and
combining the cannabis extract at the percentage concentration with an essential or carrier oil to produce the cannabis oil composition.

2. The method of claim 1, wherein the cannabis plant material is selectable from a non-exhaustive group consisting of cannabis indica plant, ruderalis plant, sativa plant and/or a hybrid plant.

3. The method of claim 1, wherein the solvent is selectable from a non-exhaustive group consisting of chloroform, light ether, ethanol isopropyl alcohol, methanol, methylene chloride, benzene and denatured ethanol.

4. The method of claim 1, wherein the predetermined amount of time comprises a range of between 0.5 hours and 8 hours.

5. The method of claim 1, wherein the predetermined amount of time is determined according to a type of solvent used.

6. (canceled)

7. The method of claim 1, wherein the step of evaporating the solvent via condensing is performed at a temperature of between 72° C. and 78° C.

8. The method of claim 1, wherein the step of performing the redox reaction comprises adding colloidal silver water at at least 40 ppm to the cannabis extract to extend said redox reaction and/or determine said percentage concentration of the cannabis extract.

9. The method of claim 1, wherein the redox reaction is performed on the cannabis extract to have a pH value of between 7.2 and 8.4.

10. The method of claim 9, wherein the essential or carrier oil comprises hemp seed oil.

11. The method of claim 1, wherein the hemp seed oil is combined with the cannabis extract to be present in the cannabis oil composition in an amount between 30% (w/w) to 95% (w/w).

12. The method of claim 1, wherein the percentage concentration of the cannabis extract is combined with the essential or carrier oil to be present in the cannabis oil composition in an amount between 7% (w/w) to 70% (w/w).

13. The method of claim 1, which comprises the step of converting the CBD (Cannabidiol) in the cannabis extract back to THC (Tetrahydrocannabinol) through isomerization.

14. The method of claim 1, wherein the method comprises the step of removing stems and/or seeds from the dried cannabis plant material before the step of eluting within a solvent.
